(19) United States
(12) Patent Application Publication
(10) Pub. No.: US 2004/0126700 A1
(43) Pub. Date: Jul. 1, 2004

(54) Ethlenic compound and structure and fabrication method of high density blue laser storage media using thereof

(76) Inventors: Ming-Chia Lee, Taichung Hsien (TW); Wen-Yih Liao, Taichung City (TW); Chien-Liang Huang, Taoyuan (TW); Chuen-Fuw Yan, Kaohsiung (TW); Tzuan-Ren Jeng, Hsinchu (TW); Andrew Teh Hu, Hsinchu (TW); Chien-Wen Chen, Pingtung (TW); Lung-Chang Liu, Shinjuang City (TW)

Correspondence Address: J.C. Patents, Inc., 4 Venture, Suite 250, Irvine, CA, 92618, US

(21) Appl. No.: 10/405,259

(22) Filed: Apr. 1, 2003

(30) Foreign Application Priority Data
Dec. 31, 2002 (TW) 91137970

(51) Int. Cl. G11B 7/24; C07C 255/00; C07C 69/00

(52) U.S. Cl. 430/270.15; 430/281.1; 430/945; 558/462; 560/130

(57) ABSTRACT

The invention provides an ethylenic compound and a high density blue ray storage media using thereof. The ethylenic compound comprises an ethylenic derivative having a chemical structures (1) as shown below.

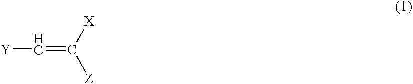

### CROSS-REFERENCE TO RELATED APPLICATION

This application claims the priority benefit of Taiwan application serial no. 91137970, filed Dec. 31, 2002.

### BACKGROUND OF THE INVENTION

1. Field of the Invention

The present invention relates to an optical storage media. More particularly, the present invention relates to an ethylenic compound, and a structure and a fabrication method of high density blue laser storage media using thereof.

2. Description of the Related Art

With the rapid advancement of internet and the development of computer capabilities, a variety of information which are available in multiplicity can be retrieved and stored. The processing speed of the computer is promoted from the condition of processing only numbers during beginning era of the invention of the computer, later advancing to the condition of processing of text, graphics, sounds, static pictures and high quality motion pictures step by step. And the information storage media for storing the information developed from using paper tape in the early days, to using magnetic tape, hard disc and a series of optical storage media developed up to the present time, such as compact disc (“CD”) and digital versatile disc (“DVD”).

The advantage of using an optical storage media as a storage media is that the optical storage media has a higher storage density than a magnetic tape. Further, low price and high writing speed of the optical storage media paved a rapid growth of the electronics market substantially. In order to satisfy the demands of the consumers, film industry and computer industry, one of the United States TV broadcaster tried to broadcast HDTV programs in the fall of 1998. This information implies that the consumers demand for a higher capacity storage media for storing motion picture so that one may be able to enjoy special video and audio effects equivalent of HDTV. For a HDTV film with duration of about 133 minutes, a storage capacity of 15 GB/side is just enough. Because the capacity of a conventional DVD disc is not enough for satisfying the requirement of enjoying special video and audio effects of HDTV for next generation, consequently, a disc driver system with higher storage capacity can be regarded an alternative solution. At present, some principles and methods of enhancing the storage density have been investigated, in that, a method is to shift the wavelength of the laser source of the optical pick-up head to a shorter wavelength range, i.e., to shorten the wavelength of the laser source. In recent years, the blue laser source of Gallium Nitride (“GaN”) series has been successfully developed, where the beam of the blue laser is more tinier by means of a higher numerical aperture (“NA”) optical lens, thus the storage density in a unit area is substantially enhanced. In order to apply the disc system using the blue laser, Sony and Philips set forth a specification for a high density blue laser disc (Blu-ray disc structure) in common. In order to fit high NA (0.85), the design of the disc structure of the high density blue laser disc is different from that of the conventional disc. As shown in FIG. 1, first of all, a reflective layer 102 is sputtered on a substrate of a thickness of about 1.1 mm, then a recording layer 104, comprised of, for example, an organic or an inorganic material, is formed on the reflective layer 102, finally a cover layer 106 of a thickness about 0.1 mm is formed over the recording layer 104. In a read operation, the laser beam emitted from the optical pick-up head of the laser is no longer read by the substrate 100, but read from the surface of the cover layer 106.

### SUMMARY OF THE INVENTION

This invention provides an ethylenic compound, and a structure and a fabrication method of high density blue laser storage media using thereof. The ethylenic compound of the present invention has a high sensitivity for the blue laser source. Further, the ethylenic compound of the present invention has an excellent solubility in organic solvents, therefore, the ethylenic compound can easily dissolve in organic solvents to form a homogenous solution and by using the homogenous solution, excellent quality film coat can be formed on the metal surface. Thus, the present invention provides a simple means and a process for making a Blu-ray disc structure.

As embodied and broadly described herein, the present invention provides an ethylenic compound comprising the following chemical structure (1): 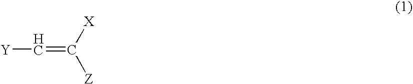 COOR₁ (2)

COOR₂ (3)

wherein the substituent X may be one selected from a group comprising hydrogen atom, cyano group, aceto-ester group, methoxycarbonyl group, wherein the aceto-ester group comprises a chemical structure (2) and wherein R₁ may be one selected from a group comprising alkyl groups with carbon number one to eight (C₁₋₈), wherein the substituent Z may be one selected from a group comprising hydrogen atom, cyano group, aceto-ester group, methoxycarbonyl group, wherein the aceto-ester group comprises a chemical structure (3) and wherein R₂ may be one selected from a group comprising alkyl groups with carbon number one to eight (C, ₁₋₈). The substituents Y are selected from a compound with or without substituents, in which the compound including monocyclic aromatic hydrocarbon group, polycyclic aromatic hydrocarbon group, heterocyclic group and ferrocenyl group, and in which the substituents are of the same or different groups selected from hydrogen atom, halogen atom, alkyl groups with carbon number one to eight (C₁₋₈), alkoxy groups with carbon number one to eight, carboxyl groups with carbon number one to eight, amino groups, amino groups with substituents, alkylate groups with carbon number one to eight, phenylate group, carboxyl group, nitro group, adamantly group, azo group, aryl group, aryloxy group, arylcarbonyl group, aryloxycarbonyl group, arylcarbonyloxy group, aryloxycarboxy group, alkylcarbonyl group, alkylcarbonyloxy group, alkoxycarbonyl group, carbamoyl group, cyanate group, cyano group, formyl group, formyloxy group, heterocyclic group, isothiocyanate group, isocyano group, isocyanate group, nitroso group, perfluoroalkyl group, perfluoroalkoxy group, sulfinyl group, fulfonyl group, silyl group, thiocyanate group and ferrocenyl group.

The ethylenic compound of the present invention has a very high sensitivity to a conventional short wavelength blue laser (with a wavelength of 405 nm). And, the ethylenic compound of the present invention with excellent solubility in organic solvents is very advantageous to the spin coating process. Moreover, the ethylenic compound of the present invention is easily synthesized at low-cost Further, the maximum absorbance of the ethylenic compound of the present invention can be easily altered by modifying the chemical structure.

The present invention provides a high density blue laser storage media, which can be at least constructed by a first substrate, a recording layer and a cover layer. The first substrate is a transparent substrate forming a signal surface. The recording layer is formed on the signal surface of the first substrate, wherein the material of the recording layer is comprised of the ethylenic compound of the present invention. The cover layer is formed over the recording layer.

In the high density blue laser storage media of the present invention, a dielectric layer is formed between the cover layer and the recording layer, a reflective layer is formed between the first substrate and the recording layer. Alternatively, a second substrate is formed instead of the cover layer, and a reflective layer is formed between the second substrate and the recording layer. The material of the dielectric layer may be comprised of, but not limited to, zinc sulfide-silicon dioxide (“ZnS—SiO2”), zinc sulfide (“ZnS”), aluminum nitride (“AlN”), silicon nitride (“SiN”) or Silica aerogel. The material of the reflective layer may be comprised of, but not limited to, gold, silver, aluminum, silicon, copper, alloy of silver and titanium, alloy of silver and chromium, alloy of silver and copper or some other alloy materials.

The present invention provides a fabrication method for manufacturing a high density blue laser storage media using the ethylenic compound of the present invention. In the method, a first transparent substrate having a signal surface is provided. A compound comprised of ethylenic compound of the present invention having chemical structure (1) is dissolved in a solvent to obtain a dye solution. Then the dye solution is coated on the first transparent substrate. Then the resulting structure is baked, and after a baking process, coated layer will be transformed into a recording layer, and then, a cover layer is formed over the storage layer.

In the fabrication method for manufacturing the high density blue laser storage media of the invention, a dielectric layer can be formed on the recording layer before the step of forming the cover layer, or a reflective layer is formed on the recording layer before the steps of forming the cover layer, and a second substrate being instead of the cover layer. The method of adhering the second substrate may include spin coating, screen printing, hot melt glue coating or double sided tape adhesion. The method of coating the dye solvent on the first transparent substrate including spin coating method, roll-pressing coating method, dip coating method or inkjet printing method.

The high density blue laser storage media of the present invention is highly sensitive to short wavelength laser, in which the wavelength is less than 500 nm for saving and loading operations. And the ethylenic compound of the present invention is highly sensitive to the light wavelength of 405 nm. Further, ethylenic compound of the present invention has an excellent solubility in organic solvents, therefore, the ethylenic compound can easily dissolve in organic solvents to form a homogenous solution and by using the homogenous solution, excellent quality film coat can be formed on the metal surface, and thus increasing the workability. Furthermore, the ethylenic compound of the present invention is easily synthesized at low-cost, and besides, the light absorbance characteristics of the ethylenic compound of the present invention can easily be altered by altering the chemical structure.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 illustrates a sectional view of a reading operation system of a disc;

FIG. 2 illustrates an ultraviolet (“UV”)/visible absorption spectrum for the compound EC-2;

FIG. 3 illustrates a thermal gravity analysis (“TGA”) graph for the compound EC-2;

FIG. 4 illustrates an UV/visible spectrum for the compound EC-10;

FIG. 5 illustrates a TGA graph for the compound EC-10;

FIG. 6 illustrates an UV/visible absorption spectrum for the compound EC-11;

FIG. 7 illustrates a TGA graph for the compound EC-11;

FIG. 8 illustrates a sectional view of a structure of a storage media according to a preferred embodiment of the present invention;

FIG. 9 illustrates a sectional view of a structure of a storage media according to another preferred embodiment of the present invention;

FIG. 10 illustrates a sectional view of a structure of a storage media according to yet another preferred embodiment of the present invention;

FIG. 11 illustrates a sectional view of a structure of a storage media according to yet another preferred embodiment of the present invention;

FIG. 12 illustrates a sectional view of a structure of a storage media according to yet another preferred embodiment of the present invention; and

FIG. 13 illustrates a reflectivity spectrum of a disc fabricated from the compound EC-11.

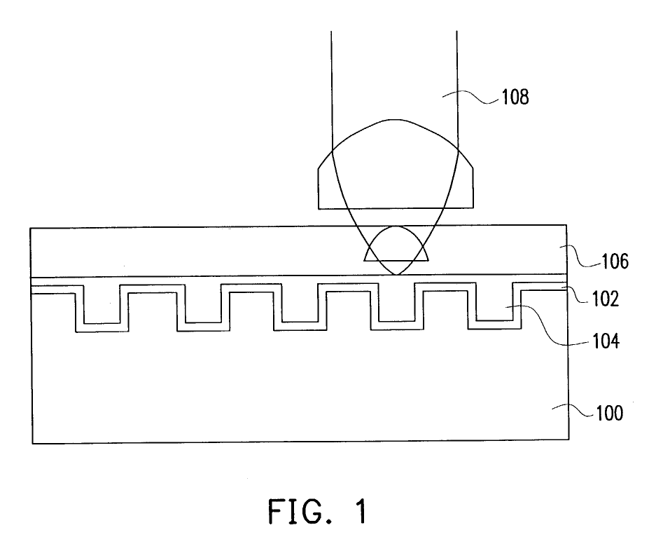

### DESCRIPTION OF THE PREFERRED EMBODIMENTS

The present invention provides a high density blue laser storage media having ethylenic compound. The ethylenic compound of the present invention comprises the following chemical structure (1): 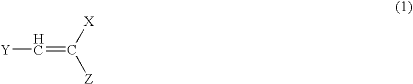 COOR₁ (2)

COOR₂ (3)

The substituent X may be selected one from a group consisting hydrogen atom, cyano group, aceto-ester group, or methoxycarbonyl group or a group having a chemical structure (2), and wherein the subsituent R₁ comprise, but limited to an alkyl groups with carbon number one to eight, the substituent Z may be selected one from a group consisting hydrogen atom, cyano group, aceto-estergroup, or methoxycarbonyl group or a group having a chemical structure (3), and wherein the subsituent R₂ comprise, but limited to an alkyl groups with carbon number one to eight. The substituents Y may be comprised of a compound with or without substituents, in which the compound including monocyclic aromatic hydrocarbon group, polycyclic aromatic hydrocarbon group, heterocyclic group and ferrocenyl group, and in which the substituents are of the same or different groups selected from hydrogen atom, halogen atom, alkyl groups with carbon number one to eight, alkoxy groups with carbon number one to eight, carboxyl groups with carbon number one to eight, amino groups, amino groups with substituents, alkylate groups with carbon number one to eight, phenylate group, carboxyl group, nitro group, adamantly group, azo group, aryl group, aryloxy group, arylcarbonyl group, aryloxycarbonyl group, arylcarbonyloxy group, aryloxycarboxy group, alkylcarbonyl group, alkylcarbonyloxy group, alkoxycarbonyl group, carbamoyl group, cyanate group, cyano group, formyl group, formyloxy group, heterocyclic group, isothiocyanate group, isocyano group, isocyanate group, nitroso group, perfluoroalkyl group, perfluoroalkoxy group, sulfinyl group, fulfonyl group, silyl group, thiocyanate group and ferrocenyl group.

The following is a description of the synthetic method for manufacturing the ethylenic compound of the present invention. The synthetic of ethylenic compound comprises a dehydration reaction of a starting material including aldehyde group with malononitrile or malonic acid dimetyl ester.

First of all, a compound having the chemical structures (4), (5) or (6):

Y—CHO (4)

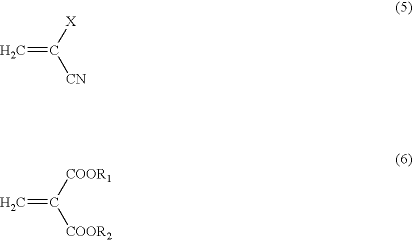

is dehydrated in an organic solution of ethylenic compound. In the chemical structures (4), (5) and (6), the substituent X is one selected from a group consisting hydrogen atom, cyano group, or methoxycarbonyl group, the subsituents R1 and R2 may be comprised of same or different chemical groups including alkyl groups with carbon number one to eight. The substituents Y may be selected from a compound with or without substituents, in which the compound including monocyclic aromatic hydrocarbon group, polycyclic aromatic hydrocarbon group, heterocyclic group and ferrocenyl group, and in which the substituents are of the same or different groups selected from hydrogen atom, halogen atom, alkyl groups with carbon number one to eight, alkoxy groups with carbon number one to eight, carboxyl groups with carbon number one to eight, amino groups, amino groups with substituents, alkylate groups with carbon number one to eight, phenylate group, carboxyl group, nitro group, adamantly group, azo group, aryl group, aryloxy group, arylcarbonyl group, aryloxycarbonyl group, arylcarbonyloxy group, aryloxycarboxy group, alkylcarbonyl group, alkylcarbonyloxy group, alkoxycarbonyl group, carbamoyl group, cyanate group, cyano group, formyl group, formyloxy group, heterocyclic group, isothiocyanate group, isocyano group, isocyanate group, nitroso group, perfluoroalkyl group, perfluoroalkoxy group, sulfinyl group, fulfonyl group, silyl group, thiocyanate group and ferrocenyl group.

Next, the ethylenic compound of the present invention are synthesized by a dehydration reaction, in which the dehydration chemical reaction is as follows:

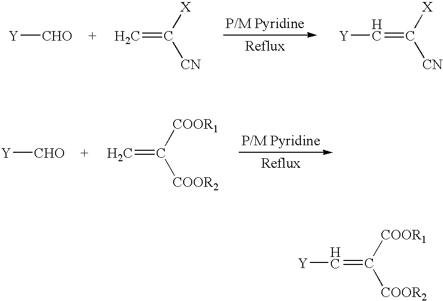

Now, the experimental examples 1 to 13 will be described in the following for describing the present invention, but however, the claims of the present invention are not limited to the experimental example 1 to 13.

### EXAMPLE 1

The starting materials 4-(dimethylamino)benzaldehyde 0.01 mole, malononitrile 0.01 mole and pyridine 1.5 g are dissolved in 10 ml of propylene glycol monomethyl ether (“PM”), and heated to the reflux temperature of the PM for 8 hours. When the reaction is completed, an orange solid is obtained. The orange solid is vacuum dried by heat, an yield of about 70% is achieved. The chemical structure of the orange solid EC-1 is shown below:

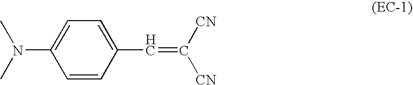

A sample of the compound EC-1 in alcohol is run in an ultraviolet (“UV”)/visible spectrometer, the maximum absorption is observed at a wavelength 434 nm.

### EXAMPLE 2

The starting materials 4-methoxybenzaldehyde 0.0 mole, malononitrile 0.01 mole and pyridine 1.5 g are dissolved in 10 ml of PM, and heated to the reflux temperature of the PM for 8 hours. When the reaction is complete, a yellow solid is obtained. The yellow solid is vacuum dried by heat, an yield of about 75% is achieved. The chemical structure of the yellow solid EC-2 is shown below:

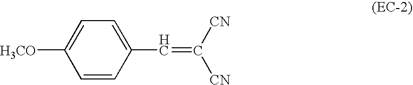

A sample of the compound EC-2 in alcohol is run in an UV/visible spectrometer, the maximum absorption is observed at a wavelength 347 nm.

### EXAMPLE 3

The starting materials ferrocenecarboxaldehyde 0.01 mole, malononitrile 0.01 mole and pyridine 1.5 g are dissolved in 10 ml of PM, and heated to the reflux temperature of the PM for 8 hours. When the reaction is complete, a dark brown solid is obtained. The dark brown solid vacuum dried by heat, an yield of about 72% is achieved. The chemical structure of the dark brown solid EC-3 is shown below:

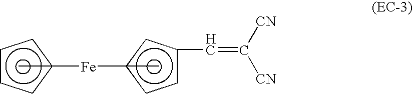

A sample of the compound EC-3 in alcohol is run in an UV/visible spectrometer, the maximum absorption is observed at a wavelength 341 nm.

### EXAMPLE 4

The starting materials 4-chlorobenzaldehyde 0.01 mole, malononitrile 0.01 mole and pyridine 1.5 g are dissolved in 10 ml of PM, and heated to the reflux temperature of the PM for 8 hours. When the reaction is complete, a brown solid is obtained. The brown solid is vacuum dried by heat, an yield of about 79% is achieved. The chemical structure of the brown solid EC-4 is shown below:

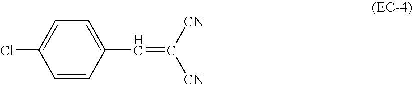

A sample of the compound EC-4 in alcohol is run in an UV/visible spectrometer, the maximum absorption is observed at a wavelength of 316 nm.

### EXAMPLE 5

The starting materials 4-tert-butylbenzaldehyde 0.01 mole, malononitrile 0.01 mole and pyridine 1.5 g are dissolved in 10 ml of PM, and heated to the reflux temperature of the PM for 8 hours. When the reaction is complete, a brown solid is obtained. The brown solid is vacuum dried by heat, an yield of about 65% is achieved. The chemical structure of the brown solid EC-5 is shown below:

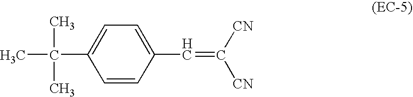

A sample of the compound EC-5 in alcohol is run in an UV/visible spectrometer, the maximum absorption is observed at a wavelength of about 323 nm.

### EXAMPLE 6

The starting materials 4-(1-pyrrolidino)benzaldehyde 0.01 mole, malononitrile 0.01 mole and pyridine 1.5 g are dissolved in 10 ml of PM, and heated to the reflux temperature of the PM for 8 hours. When the reaction is completed, an orange solid is obtained. The orange solid is vacuum dried by heat, an yield of about 60% is achieved. The chemical structure of the orange solid EC-6 is shown below:

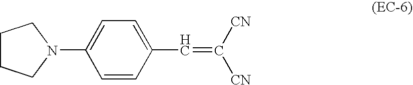

A sample of the compound EC-6 in alcohol is run in an UV/visible spectrometer, the maximum absorption is observed at a wavelength of about 434 nm.

### EXAMPLE 7

The starting materials 4-ethylbenzaldehyde 0.01 mole, malononitrile 0.01 mole and pyridine 1.5 g are dissolved in 10 ml of PM, and heated to the reflux temperature of the PM for 8 hours. When the reaction is complete, a light brown solid is obtained. The light brown solid is vacuum dried by heat, an yield of about 63% is achieved. The chemical structure of the light brown solid EC-7 is shown below:

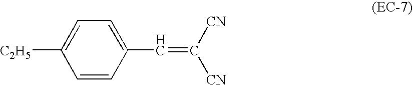

A sample of the compound EC-7 in alcohol is run in an UV/visible spectrometer, the maximum absorption is observed at a wavelength of about 323 nm.

### EXAMPLE 8

The starting materials N-ethyl-3-carbazolecarboxaldehyde 0.01 mole, malononitrile 0.01 mole and pyridine 1.5 g are dissolved in 10 ml of PM, and heated to the reflux temperature of the PM for 8 hours. When the reaction is complete, a yellowish brown solid is obtained. The yellowish solid is vacuum dried by heat, an yield of about 78% is achieved. The chemical structure of the yellowish brown solid EC-8 is shown below.

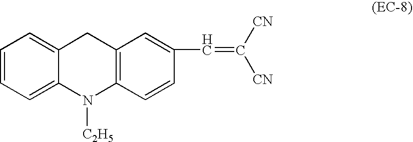

A sample of the compound EC-8 in alcohol is run in an UV/visible spectrometer, the maximum absorption is observed at a wavelength of about 407 nm.

### EXAMPLE 9

The starting materials 1-adamantanecarbonyl chloride 0.01 mole, 2-[(4-hydroxyphenyl)methylene]malononitrile 0.01 mole and sodium acetate 1 g are dissolved in 20 ml of PM, and heated to the reflux temperature of the PM for 8 hours. When the reaction is complete, a dark brown solid is obtained. The dark brown solid is vacuum dried by heat, an yield of about 56% is achieved. The chemical structure of the dark brown solid EC-9 is shown below:

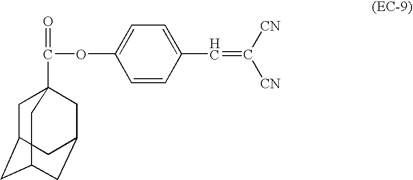

A sample of the compound EC-9 in alcohol is run in an UV/visible spectrometer, the maximum absorption is observed at a wavelength of about 354 nm.

### EXAMPLE 10

The starting materials 4-(dimethylamino)benzaldehyde 0.01 mole, malonic acid dimethyl ester 0.01 mole and pyridine 1.5 g are dissolved in 10 ml of PM, and heated to the reflux temperature of the PM for 8 hours. When the reaction is complete, a yellow solid is obtained. The yellow solid is concentrated, an yield of about 55% is achieved. The chemical structure of the yellow solid EC-10 is shown below:

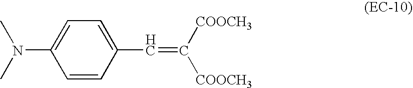

A sample of the compound EC-10 in alcohol is run in an UV/visible spectrometer, the maximum absorption is observed at a wavelength of about 338 nm.

### EXAMPLE 11

The starting materials 4-(1-pyrrolidino)benzaldehyde 0.01 mole, malonic acid dimethyl ester 0.01 mole and pyridine 1.5 g are dissolved in 10 ml of PM, and heated to the reflux temperature of the PM for 8 hours. When the reaction is complete, an orange solid is obtained. The orange solid is concentrated, an yield of about 62% is achieved. The chemical structure of the orange solid EC-11 is shown below:

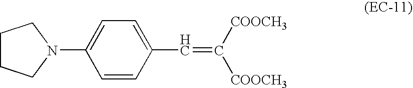

A sample of the compound EC-11 in alcohol is run in an UV/visible spectrometer, the maximum absorption is observed at a wavelength of about 345 nm.

### EXAMPLE 12

The starting materials 4-anthraldehyde 0.01 mole, malonic acid dimethyl ester 0.01 mole and pyridine 1.5 g are dissolved in 10 ml of PM, and heated to the reflux temperature of the PM for 8 hours. When the reaction is completed, a yellow solid is obtained. The yellow solid is concentrated, an yield of about 58% is achieved. The chemical structure of the yellow solid EC-12 is shown below:

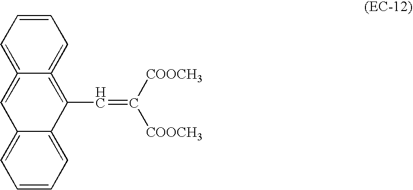

A sample of the compound EC-12 in alcohol is run in an UV/visible spectrometer, the maximum absorption is observed at a wavelength of about 401 nm.

### EXAMPLE 13

The starting materials 4-dimethylaminobenzaldehyde 0.01 mole, malonic acid dimethyl ester 0.01 mole and pyridine 1.5 g are dissolved in 10 ml of PM, and heated to the reflux temperature of the PM for 8 hours. When the reaction is complete, a yellow solid is obtained. The yellow solid is concentrated, an yield of about 53% is achieved. The chemical structure of the yellow solid EC-13 is shown below:

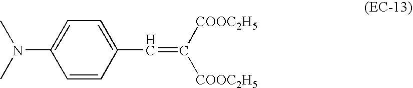

A sample of the compound EC-13 in alcohol is run in an UV/visible spectrometer, the maximum absorption is observed at a wavelength of about 401 nm.

The eethylenic compounds obtained from the processes described in examples 1 to 13 is subjected to purification, e.g., by re-crystallizing the ethylenic compounds in alcohol, wherein the re-crystallization is carried out in a condition where a ratio of the ethylenic compounds to the alcohol is in a range of about 0.05 to 0.1.

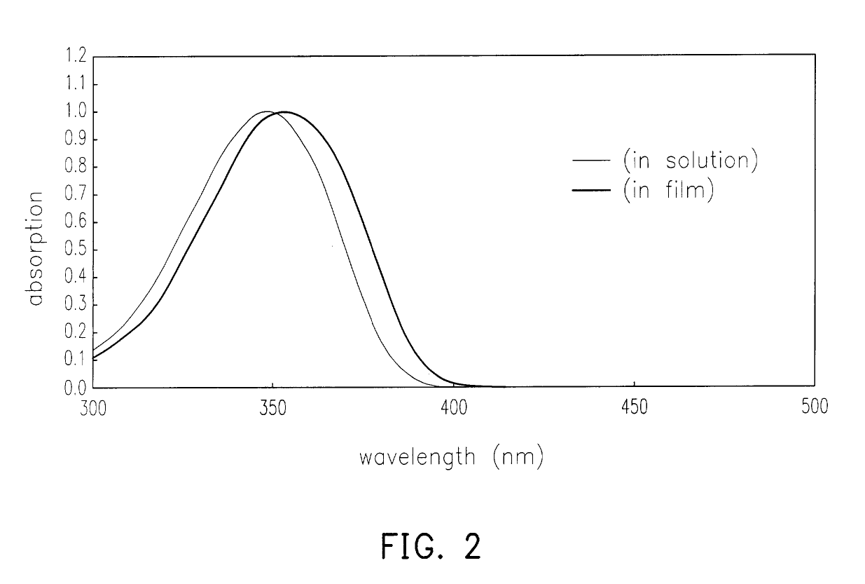

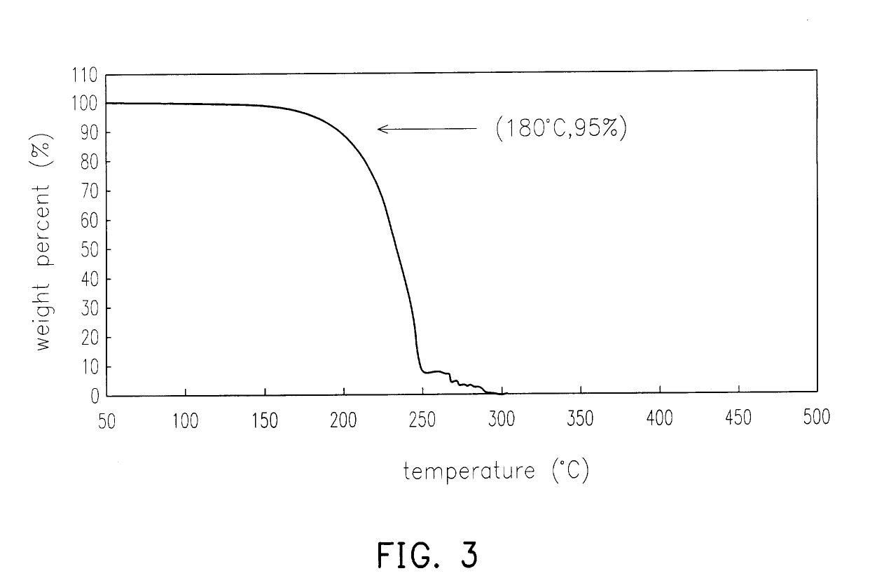

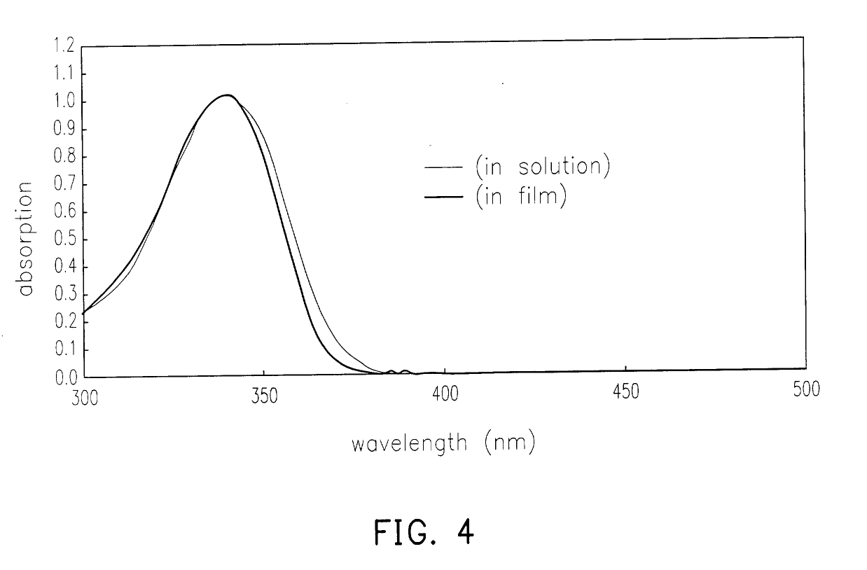

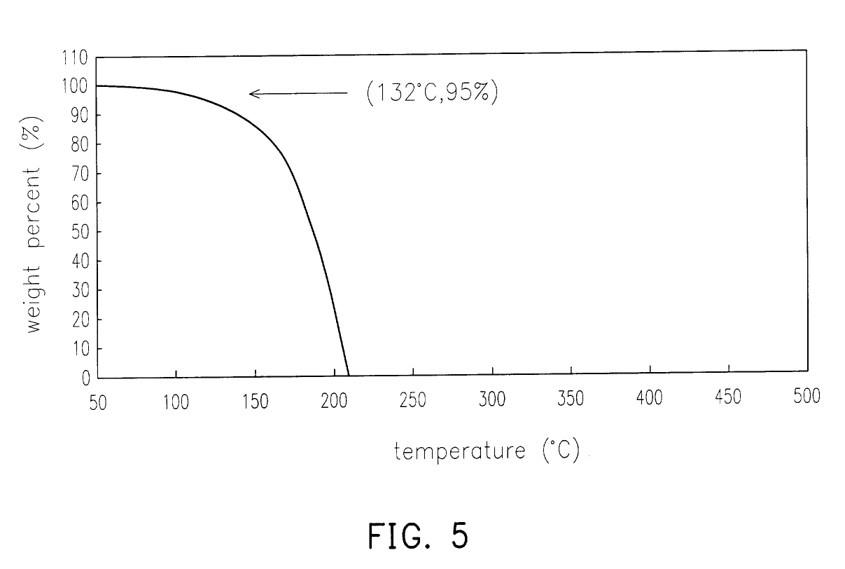

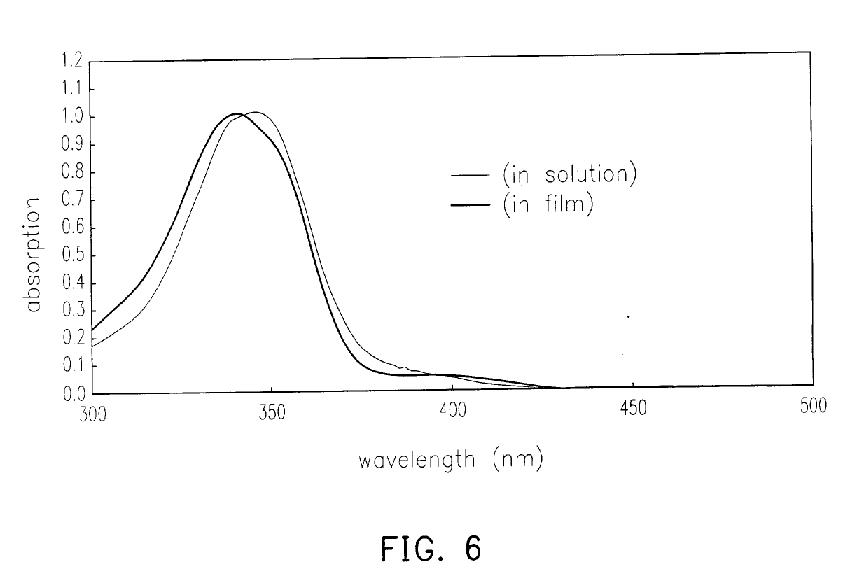

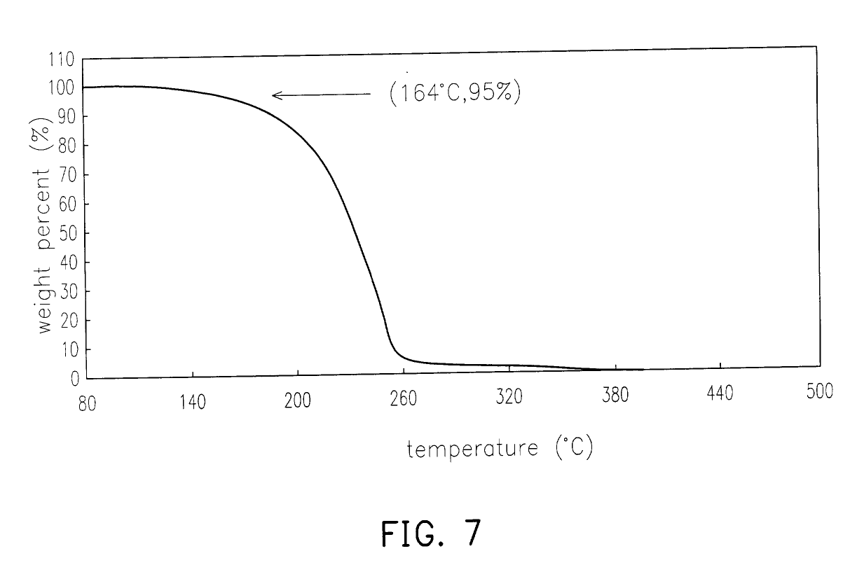

The synthesized ethylenic compounds obtained from the process described in examples 1 to 13 are measured by a thermal gravity analyzer (“TGA”) and an UV/visible spectrometer, and the thermal degradation temperature (i.e., the temperature of 5 wt % thermal weight loss) and the spectroscopic property (e.g., the maximum absorption wavelength of the thin film or the solution) are measured. The results are listed in Table 1 below. In Table 1, the UV/visible spectrum and the profile of TGA of the compound EC-2 of the example 2 are shown in FIGS. 2 and 3 respectively. The UV/visible spectrum and the profile of TGA of the compound EC-10 of the example 10 are shown in FIGS. 4 and 5 respectively. The UV/visible spectrum and the profile of TGA of the compound EC-11 of the example 11 are shown in FIGS. 6 and 7 respectively.

TABLE I

| | Maximum absorption λₘₐₓ (nm) | | |
|---|---|---|---|
| Ethylenic | Solution (in | | Thermal degradation |
| compounds | alcohol) | Thin film | temperature (° C.) |
| EC-1 | 434 | 441 | 232 |
| EC-2 | 347 | 352 | 180 |
| EC-3 | 341 | 346 | 230 |
| EC-4 | 316 | 321 | 170 |
| EC-5 | 323 | 327 | 152 |
| EC-6 | 434 | 440 | 260 |
| EC-7 | 323 | 327 | 162 |
| EC-8 | 407 | 416 | 277 |
| EC-9 | 354 | 355 | 171 |
| EC-10 | 338 | 340 | 132 |
| EC-11 | 345 | 341 | 164 |
| EC-12 | 401 | 409 | 205 |
| EC-13 | 338 | 339 | 140 |

The results of Table 1 infer that the ethylenic compound of the present invention is suitable for manufacturing a storage media for the saving and loading operations using the short wavelength laser with a wavelength less than 500 nm, and more particularly the short wavelength blue light laser (with a wavelength of 405 nm).

A process of spin coating a recording layer using the compound EC-11 of the present invention is described as follows. The compound EC-11 of the example 11 is dissolved in the solvent 2,2,3,3-tetrafluoropropanol, preferably a 2 wt % solution of the ethylenic compound is prepared. The solution of the ethylenic compound is spin coated on a transparent substrate, it is preferable that no land structures are formed in the transparent substrate. The refractive index n of the coated layer measured is about 1.7, and the dielectric constant k of the coated layer is about 0.08.

The ethylenic compound ethylenic compound of the present invention is very sensitive to the short wavelength blue laser (with a wavelength of 405 nm). As the ethylenic compounds are highly soluble in organic solvents, they have the capability to form excellent quality spin-coated layers on a metal surface using the spin coating process. Moreover, the ethylenic compounds are easily synthesized at low-cost. Further as the maximum absorption wavelength of that is easily altered by altering the chemical structure, makes the application of the ethylenic compound of the present invention to be more comprehensive.

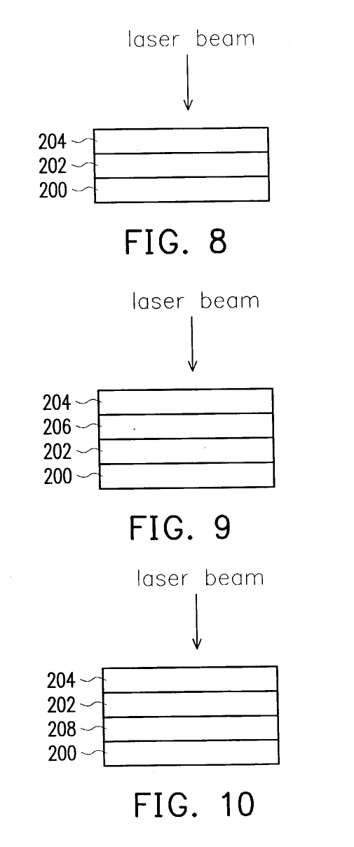

A fabrication method for manufacturing a high density storage media using the ethylenic compounds of the present invention will be described in the following examples 14 to 18. FIGS. 8 to 12 illustrates the structures of the high density blue laser storage media by using the fabrication methods described in the experimental examples 14 to 18.

### EXAMPLE 14

Referring to FIG. 8, a substrate 200, such as a transparent substrate having lands or grooves, pits and no recording data, is provided. The lands or pre-carved pits included in the substrate 200 provide a signal surface for the laser tracking of the pick-up head of the laser. The material of the substrate 200 includes, such as polyster, polycarbonate, polymethylmethacrylate (PMMA), metallocene based cyclic olefin copolymers (mCOC).

The ethylenic compound of the present invention is dissolved in an organic solvent or a dye-in-polymer solution. The organic solvent is comprised of, but not limited to a alcohol with carbon number one to six, a ketone with carbon number one to six, an ether with carbon number one to six, a dibutyl ether (“DBE”), halogen compounds, an amide or a methylcyclohexane (“MCH”). Examples of alcohol with carbon number one to six include but not limited to, such as, methanol, ethanol, isopropanol, diacetonalchol (“DAA”), ether alcohol with carbon number one to six, propylene glycol monoethyl ether, propylene glycol monoethyl acetate, 2,2,3,3-tetrafluoropropanol, trichloroethanol, 2-chloroethanol, octafluoropentanol or hexafluorobutanol. Examples of ketone with carbon number one to six include but not limited to, such as, acetone, methyl isobutyl ketone,(“MIBK”), methyl ethyl ketone, (“MEK”), or 3-hydroxy-3-methyl-2-butanone. Examples of halogen compounds include, such as chloroform, dichloromethane or 1-chlorobutane. The amide includes, such as dimethylformamide (“DHF”) or dimethylacetamide (“DMA”). Examples for polymers in the dye-in-polymer solution include but not limited to, chitin, cellulose (e.g., cellulose ester, nitrocellulose, cellulose acetate, cellulose acetate butyrate and so forth), polyvinyl butyral and so forth.

The above solution containing the ethylenic compound of the present invention is coated on the substrate 200, by using a coating method, including but not limited to, for example, a spin coating method, a roll-pressing method, a dip coating method and an inkjet printing method. Next, the resulting structure is subjected to a baking process to form a recording layer 202 ethylenic compound over the substrate 200.

Finally, a cover layer 204 with a thickness of about 0.1 mm, is coated over the storage layer 202, thus, the fabrication of a high density blue laser storage media is completed.

By using the fabrication method of a disc described above, and taking the compound EC-11 of the present invention as a specific example, the EC-11 is dissolved in 2,2,3,3-tetrafluoropropanol, preferably a 2 wt % solution ethylenic compound is prepared. The above solution ethylenic compound is spin coated on a substrate 200, wherein the substrate 200 comprises land having a depth of about 30 mm, and track pitch of about 0.35 um after the resulting structure is subjected to a baking process to form a recording layer 202. Finally a cover layer 204 with a thickness of about 0.1 mm is coated on the recording layer 202. Thus, a high density blue laser storage media is completed. The reflectivity of the disc produced from the above process is about 10%.

### EXAMPLE 15

Referring to FIG. 9, a substrate 200, comprising lands or pre-curved pits, is provided. The ethylenic compound of the present invention is dissolved in an organic solvent or an ethylenic compound-in-polymer solution to form a solution. The above solution is coated on the substrate 200. The resulting structure is subjected to a baking process to form a recording layer 202 comprised of ethylenic compound over the substrate 200. Next, a dielectric layer 206 is formed on the recording layer 202. The material of the dielectric layer 206 is comprised of, but not limited to, ZnS—SiO2, ZnS, AlN, SiN or Silica aerogel. Finally a cover layer 204 with a thickness of about 0.1 mm is coated on the dielectric layer 206. Thus, the fabrication of a high density blue laser storage media is completed.

By using the fabrication method of a disc described above, and taking the compound EC-11 of the present invention as a specific example, the EC-11 is dissolved in 2,2,3,3-tetrafluoropropanol, preferably, a 2 wt % solution of ethylenic compound EC-11 is prepared. The solution of the ethylenic compound is spin coated on a substrate 200 comprising land having a depth of about 30 mm, and track pitch of about 0.35 um. The resulting structure is subjected to a baking process to form a storage layer 202. Next, a cover layer 204 with a thickness of about 0.1 mm is coated on the recording layer 202. Thus, the fabrication of a high density blue laser storage media is completed. The reflectivity of the disc produced using the above process is about 15%.

### EXAMPLE 16

Referring to FIG. 10, a substrate 200, comprising lands or pre-curved pits, is provided. The lands or pre-curved pits included in the substrate 200 provide a signal surface for the laser tracking of the pick-up head of the laser. Next, a reflective layer 208 is formed on the substrate 200. The material of the reflective layer 208 comprised of, but not limited to, gold, silver, aluminum, silicon, copper, alloy of silver and titanium, alloy of silver and chromium, alloy of silver and copper or some other alloy materials. The ethylenic compound of the present invention is dissolved in an organic solvent or a dye-in-polymer solution to form a solution. The above solution is coated on the reflective layer 208 using a conventional method, such as spin coating method, roll-pressing method, dip coating method and inkjet printing method. After the resulting structure is subjected to a baking process to form a recording layer 202 comprised of ethylenic compound over the reflective layer 208. Finally a cover layer 204 with a thickness, such as 0.1 mm, is coated on the dielectric layer 206. Thus, the fabrication of a high density blue laser storage media is completed.

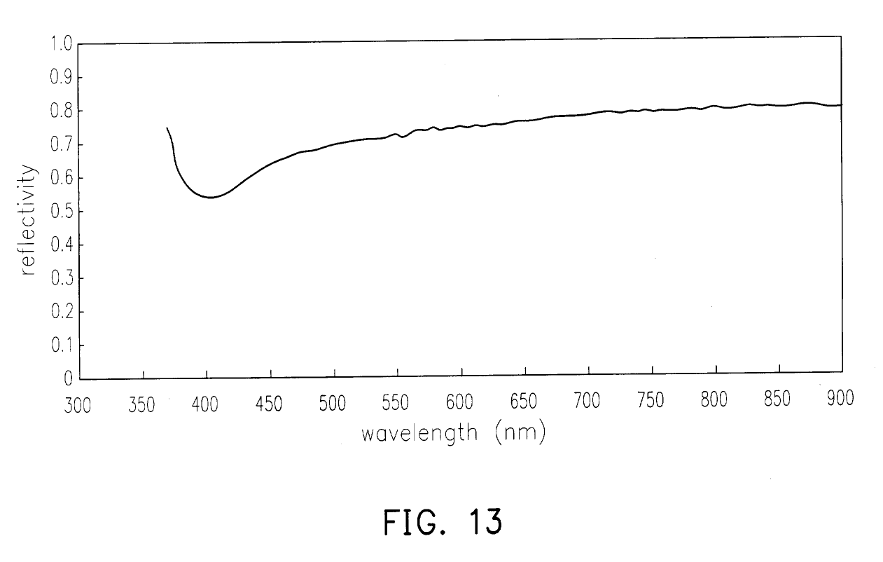

By using the fabrication method of a disc described above, and taking the compound EC-11 of the present invention as a specific example, the compound EC-11 is dissolved in 2,2,3,3-tetrafluoropropanol, preferably, a 2 wt % solution is prepared. The above solution is coated on the reflective layer 208 which is comprised of a layer is silver with a thickness of about 50 nm formed on the substrate 200 having land with a depth of about 30 mm, and track pitch of about 0.35 um. The resulting structure is subjected to a baking process to form a recording layer 202. Finally a cover layer 204 with a thickness of about 0.1 mm is coated on the recording layer 202. Thus, the fabrication of a high density blue laser storage media is completed. The measured reflective spectrum of the disc produced by using the above process is shown in FIG. 13, and the reflectivity of the disc is about 54% at a wavelength of 405 nm.

### EXAMPLE 17

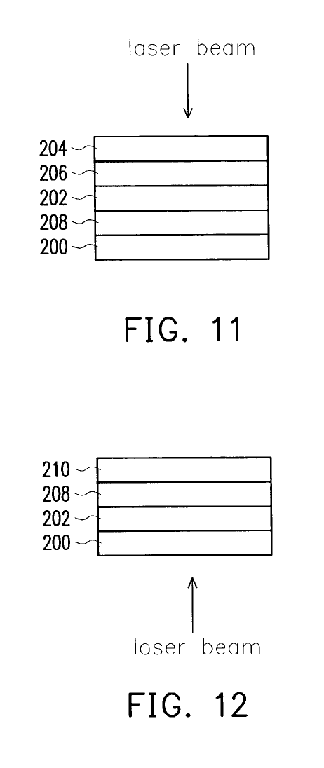

Referring to FIG. 11, a substrate 200, comprising lands or pre-curved pits, is provided. The lands or pre-curved pits in the substrate 200 provide a signal surface for the laser tracking of the pick-up head of the laser. Next, a reflective layer 208 is formed on the substrate 200. The ethylenic compound of the present invention is dissolved in an organic solvent or a dye-in-polymer solution to form a solution. The above solution containing the ethylenic compound of the present invention is coated on the reflective layer 208. The, resulting structure is then subjected to a baking process to form a recording layer 202 comprised of ethylenic compound. A dielectric layer 206 is formed on the recording layer 202. Finally a cover layer 204 with a thickness of about 0.1 mm is coated on the dielectric layer 206. Thus, the fabrication of a high density blue laser storage media is completed.

By using the fabrication method of a disc described above, and taking the compound EC-11 of the present invention as a specific example, the compound EC-11 is dissolved in 2,2,3,3-tetrafluoropropanol, preferably, a 2 wt % solution is prepared. The above solution containing the compound EC-11 is spin coated on the reflective layer 208 comprised of silver with a thickness of about 50 nm formed on the substrate 200 having lands with a depth of about 30 mm, and the track pitches of about 0.35 um. Then, the resulting structure is subjected to a baking process to form a recording layer 202. A dielectric layer 206 is formed on the recording layer 202. Finally a cover layer 204 with a thickness of about 0.1 mm is coated on the recording layer 202. Thus, the fabrication of a high density blue laser storage media is completed. The reflectivity of the above disc is about 64%.

### EXAMPLE 18

Referring to FIG. 12, a substrate 200, comprising lands or pre-curved pits, is provided. The lands or pre-curved pits in the substrate 200 provide a signal surface for the laser tracking of the pick-up head of the laser. The ethylenic compound of the present invention is dissolved in an organic solvent or a dye-in-polymer solution, and a solution of the derivative. The above solution containing the ethylenic compound of the present invention is coated on the substrate 200. The, the resulting structure is subjected to a baking process to form a recording layer 202 ethylenic compound on the substrate 200. Next, a reflective layer 208 is formed on the recording layer 202. Finally another transparent substrate 210 is adhered over the reflective layer 208 on it. Thus, the fabrication of a high density blue laser storage media is completed. The material of the transparent substrate 210 comprised of, but not limited to, polyster, polycarbonate, polymethylmethacrylate (PMMA), metallocene based cyclic olefin copolymers (mCOC). The method of adhering the substrate 200 to the reflective layer 208 include spin coating, screen printing, hot melt glue coating or double sided tape adhesion.

The high density blue laser storage media described in the present invention provides the short wavelength laser, wherein the wavelength is less than 500 nm for performing saving and loading operations. The ethylenic compound, and the high density blue laser storage media comprising the ethylenic compound of the present invention is highly sensitive to the laser wavelength of about 405 nm. Further, the ethylenic compound of the present invention is highly soluble in the organic solvent and form an excellent film on the surface the metal layer, thus, increasing the workability. Furthermore, the ethylenic compound of the present invention can be easily synthesized at a low-cost. The absorbance of the ethylenic compound of the present invention can be easily altered by altering the chemical structure, thus a recording layer with a desirable absorbance can be manufactured.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. An ethylenic compound comprises:
a ethylenic derivative, having a following chemical structure: 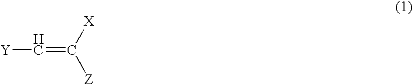
wherein the substituent X is selected from a group consisting a hydrogen atom, a cyano group, a methoxycarbonyl group or a group having a following chemical structure (2) shown below:
COOR₁ (2)
wherein the subsituent R₁ comprises an alkyl group with carbon number one to eight;
wherein the substituent is selected from a group consisting a hydrogen atom, a cyano group, a methoxycarbonyl group or a group having a following chemical structure (3) shown below:
COOR₂ (3)
wherein the subsituent R₂ comprises an alkyl group with carbon number one to eight; and
the substituents Y comprises a compound with or without substituents, wherein the compound comprising monocyclic aromatic hydrocarbon group, polycyclic aromatic hydrocarbon group, heterocyclic group and ferrocenyl group; and wherein the substituents are of the same or different groups comprising hydrogen atom, halogen atom, alkyl groups with carbon number one to eight, alkoxy groups with carbon number one to eight, carboxyl groups with carbon number one to eight, amino groups, amino groups with substituents, alkylate groups with carbon number one to eight, phenylate group, carboxyl group, nitro group, adamantly group, azo group, aryl group, aryloxy group, arylcarbonyl group, aryloxycarbonyl group, arylcarbonyloxy group, aryloxycarboxy group, alkylcarbonyl group, alkylcarbonyloxy group, alkoxycarbonyl group, carbamoyl group, cyanate group, cyano group, formyl group, formyloxy group, heterocyclic group, isothiocyanate group, isocyano group, isocyanate group, nitroso group, perfluoroalkyl group, perfluoroalkoxy group, sulfinyl group, fulfonyl group, silyl group, thiocyanate group and ferrocenyl group.

2. A high density blue laser storage media comprising:
a first transparent substrate, having a signal surface;
a recording layer formed on the signal surface of the first transparent substrate, wherein the recording layer comprises an ethylenic derivative having a chemical structure (1) shown below: 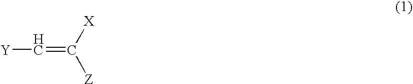
wherein the substituent X is selected from a group consisting of a hydrogen atom, a cyano group, a methoxycarbonyl group or a group having a following chemical structure (2) shown below:
COOR₁ (2)
wherein the subsituent R₁ comprises an alkyl group with carbon number one to eight;
wherein the substituent Z is selected from a group consisting a hydrogen atom, a cyano group, a methoxycarbonyl group or a group having a following chemical structure (3) shown below:
COOR₂ (3)
wherein the subsituent R₂ comprises an alkyl group with carbon number one to eight; and
the substituents Y comprises a compound with or without substituents, wherein the compound comprising monocyclic aromatic hydrocarbon group, polycyclic aromatic hydrocarbon group, heterocyclic group and ferrocenyl group; and wherein the substituents are of the same or different groups comprising hydrogen atom, halogen atom, alkyl groups with carbon number one to eight, alkoxy groups with carbon number one to eight, carboxyl groups with carbon number one to eight, amino groups, amino groups with substituents, alkylate groups with carbon number one to eight, phenylate group, carboxyl group, nitro group, adamantly group, azo group, aryl group, aryloxy group, arylcarbonyl group, aryloxycarbonyl group, arylcarbonyloxy group, aryloxycarboxy group, alkylcarbonyl group, alkylcarbonyloxy group, alkoxycarbonyl group, carbamoyl group, cyanate group, cyano group, formyl group, formyloxy group, heterocyclic group, isothiocyanate group, isocyano group, isocyanate group, nitroso group, perfluoroalkyl group, perfluoroalkoxy group, sulfinyl group, fulfonyl group, silyl group, thiocyanate group and ferrocenyl group.

3. The high density blue laser storage media of claim 2, wherein a dielectric layer is formed between the first transparent substrate and the recording layer.

4. The high density blue laser storage media of claim 3, wherein the material of the dielectric layer comprises zinc sulfide-silicon dioxide (“ZnS—SiO2”), zinc sulfide (“ZnS”), aluminum nitride (“AlN”), silicon nitride (“SiN”) or Silica aerogel.

5. The high density blue laser storage media of claim 2, wherein a reflective layer is formed between the first transparent substrate and the recording layer.

6. The high density blue laser storage media of claim 5, wherein the material of the reflective layer comprises gold, silver, aluminum, silicon, copper, alloy of silver and titanium, alloy of silver and chromium, alloy of silver and copper.

7. The high density blue laser storage media of claim 2, wherein the high density blue laser storage media further comprises:
a reflective layer, formed between the first transparent substrate and the recording layer;
a cover layer, formed over recording layer; and
a dielectric layer, formed between the cover layer and the recording layer.

8. The high density blue laser storage media of claim 7, wherein the material of the reflective layer comprises gold, silver, aluminum, silicon, copper, alloy of silver and titanium, alloy of silver and chromium, alloy of silver and copper.

9. The high density blue laser storage media of claim 7, wherein the material of the dielectric layer comprises ZnS—SiO2, ZnS, AlN, SiN or Silica aerogel.

10. The high density blue laser storage media of claim 2, wherein the high density blue laser storage media further comprises:
a second substrate, formed over the recording layer, and
a reflective layer, formed between the second substrate and the recording layer.

11. The high density blue laser storage media of claim 10, wherein the material of the second substrate comprises polyster, polycarbonate, polymethylmethacrylate (PMMA), metallocene based cyclic olefin copolymers (mCOC).

12. The high density blue laser storage media of claim 10, wherein the material of the reflective layer comprises gold, silver, aluminum, silicon, copper, alloy of silver and titanium, alloy of silver and chromium, alloy of silver and copper.

13. The high density blue laser storage media of claim 2, wherein the material of the first transparent substrate comprises polyster, polycarbonate, polymethylmethacrylate (PMMA), metallocene based cyclic olefin copolymers (mCOC).

14. A high density blue laser storage media comprising:
at least a recording layer comprising of an ethylenic derivative having a chemical structure (I) shown below: 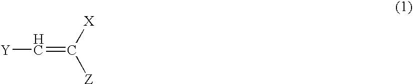
wherein the substituent X is selected from a group consisting of a hydrogen atom, a cyano group, a methoxycarbonyl group or a group having a following chemical structure (2) shown below:
COOR₁ (2)
wherein the subsituent R₁ is selected from a group consisting of an alkyl group with carbon number one to eight;
wherein the substituent Z comprises hydrogen atom, cyano group, methoxycarbonyl group or a group having a following chemical structure (3) shown below:
COOR₂ (3)
wherein the subsituent R₂ is selected from a group consisting of an alkyl group with carbon number one to eight, and
the substituents Y comprises a compound with or without substituents, wherein the compound comprising monocyclic aromatic hydrocarbon group, polycyclic aromatic hydrocarbon group, heterocyclic group and ferrocenyl group; and wherein the substituents are of the same or different groups comprising hydrogen atom, halogen atom, alkyl groups with carbon number one to eight, alkoxy groups with carbon number one to eight, carboxyl groups with carbon number one to eight, amino groups, amino groups with substituents, alkylate groups with carbon number one to eight, phenylate group, carboxyl group, nitro group, adamantly group, azo group, aryl group, aryloxy group, arylcarbonyl group, aryloxycarbonyl group, arylcarbonyloxy group, aryloxycarboxy group, alkylcarbonyl group, alkylcarbonyloxy group, alkoxycarbonyl group, carbamoyl group, cyanate group, cyano group, formyl group, formyloxy group, heterocyclic group, isothiocyanate group, isocyano group, isocyanate group, nitroso group, perfluoroalkyl group, perfluoroalkoxy group, sulfinyl group, fulfonyl group, silyl group, thiocyanate group and ferrocenyl group.

15. A fabrication method of a high density blue laser storage media, comprising the steps of:
providing a first transparent substrate, having a signal surface;
forming a solution of ethylenic compound, wherein ethylenic compound comprises a ethylenic derivative having a following chemical structure (1): 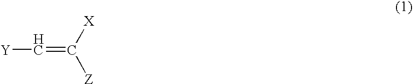
wherein the substituent X is selected from a group consisting of a hydrogen atom, a cyano group, a methoxycarbonyl group or a group having a following chemical structure (2) shown below:
COOR₁ (2)
wherein the subsituent R₁ is selected from a group consisting of an alkyl group with carbon number one to eight;
wherein the substituent Z comprises hydrogen atom, cyano group, methoxycarbonyl group or a group having a following chemical structure (2) shown below:
COOR₂ (3)
wherein the subsituent R₂ is selected from a group consisting of an alkyl group with carbon number one to eight; and
the substituents Y comprises a compound with or without substituents, wherein the compound comprising monocyclic aromatic hydrocarbon group, polycyclic aromatic hydrocarbon group, heterocyclic group and ferrocenyl group; and wherein the substituents are of the same or different groups comprising hydrogen atom, halogen atom, alkyl groups with carbon number one to eight, alkoxy groups with carbon number one to eight, carboxyl groups with carbon number one to eight, amino groups, amino groups with substituents, alkylate groups with carbon number one to eight, phenylate group, carboxyl group, nitro group, adamantly group, azo group, aryl group, aryloxy group, arylcarbonyl group, aryloxycarbonyl group, arylcarbonyloxy group, aryloxycarboxy group, alkylcarbonyl group, alkylcarbonyloxy group, alkoxycarbonyl group, carbamoyl group, cyanate group, cyano group, formyl group, formyloxy group, heterocyclic group, isothiocyanate group, isocyano group, isocyanate group, nitroso group, perfluoroalkyl group, perfluoroalkoxy group, sulfinyl group, fulfonyl group, silyl group, thiocyanate group and ferrocenyl group;
coating the ethylenic compound solution on the first transparent substrate;
performing a baking process after the coating step to form a recording layer; and
coating a cover layer on the recording layer.

16. The fabrication method of claim 15, further comprises forming a dielectric layer after the baking process, and before coating the cover layer on the recording layer.

17. The fabrication method of claim 15, wherein the material of the dielectric layer comprises ZnS—SiO2, ZnS, AlN, SiN or Silica aerogel.

18. The fabrication method of claim 15, further comprises forming a reflective layer before the step of forming the ethylenic compound solution.

19. The fabrication method of claim 18, wherein the material of the reflective layer comprises gold, silver, aluminum, silicon, copper, alloy of silver and titanium, alloy of silver and chromium, alloy of silver and copper.

20. The fabrication method of a claim 15, further comprises:
forming a reflective layer over the first transparent substrate and before the step of forming the ethylenic compound solution; and
forming a dielectric layer after the baking process, and before the step coating the cover layer on the recording layer.

21. The fabrication method of claim 20, wherein a material of the reflective layer comprises gold, silver, aluminum, silicon, copper, alloy of silver and titanium, alloy of silver and chromium, alloy of silver and copper.

22. The fabrication method of claim 20, wherein a material of the dielectric layer comprises ZnS—SiO2, ZnS, AlN, SiN or Silica aerogel.

23. The fabrication method of claim 15, further comprises:
forming a reflective layer over the recording layer after the baking process; and
adhering a second substrate on the reflective layer.

24. The fabrication method of claim 22, wherein the second substrate comprises a transparent substrate.

25. The fabrication method of claim 23, wherein a material of the reflective layer comprises gold, silver, aluminum, silicon, copper, alloy of silver and titanium, alloy of silver and chromium, alloy of silver and copper.

26. The fabrication method of claim 23, wherein the method of adhering the second substrate to the reflective layer comprises a spin coating method, a screen printing method, a hot melt glue coating method or a double sided tape adhesion method.

27. The fabrication method of claim 15, wherein the process of forming a ethylenic compound solution comprises dissolving the ethylenic derivative in an organic solvent.

28. The fabrication method of claim 27, wherein the organic solvent comprises an alcohol with carbon number one to six, a ketone with carbon number one to six, ether with carbon number one to six, a dibutyl ether (“DBE”), an halogen compound, amide or a methylcyclohexane (“MCH”).

29. The fabrication method of claim 28, wherein the alcohol with carbon number one to six comprises methanol, ethanol, isopropanol, diacetonalchol (“DAA”), ether alcohol with carbon number one to six, propylene glycol monoethyl ether, propylene glycol monoethyl acetate, 2,2,3,3-tetrafluoropropanol, trichloroethanol, 2-chloroethanol, octafluoropentanol or hexafluorobutanol.

30. The fabrication method of claim 28, wherein the ketone with carbon number one to six comprises acetone, methyl isobutyl ketone,(“MIBK”), methyl ethyl ketone, (“MEK”), or 3-hydroxy-3-methyl-2-butanone.

31. The fabrication method of claim 27, wherein the halogen compound comprises chloroform, dichloromethane or 1-chlorobutane.

32. The fabrication method of claim 28, wherein the amide comprises dimethylformamide (“DHF”) or dimethylacetamide (“DMA”).

33. The fabrication method of claim 15, wherein the process of forming the ethylenic compound solution comprises dissolving the ethylenic derivative in an dye-in-polymer solution.

34. The fabrication method of claim 33, wherein the polymers in the dye-in-polymer solution comprise chitin, cellulose or polyvinyl butyral.

35. The fabrication method of claim 15, wherein the method of coating the ethylenic compound solution on the first transparent substrate comprises a spin coating method, a roll-pressing coating method, a dip coating method or an inkjet printing method.

36. The fabrication method of claim 15, wherein a material of the first transparent substrate comprises polyster, polycarbonate, polymethylmethacrylate (PMMA) or metallocene based cyclic olefin copolymers (mCOC).
